(19) United States
(12) Patent Application Publication
(10) Pub. No.: US 2015/0293120 A1
(43) Pub. Date: Oct. 15, 2015

(54) MARKER SEQUENCES FOR RHEUMATOID ARTHRITIS

(71) Applicant: PROTAGEN AG, Dortmund (DE)

(72) Inventors: Angelika LÜKING, Bochum (DE); Peter SCHULZ-KNAPPE, Hemmingen (DE); Heike GÖHLER, Bochum (DE); Martin GAMER, Dortmund (DE); Carmen THEEK, Herdecke (DE); Daniel CHAMRAD, Bochum (DE); Anna TELAAR, Dortmund (DE); Matthias VON DARL, Steinfurt (DE); Matthias SCHNEIDER, Düsseldorf (DE); Jessica SCHWERMANN, Isernhagen (DE)

(21) Appl. No.: 14/388,533

(22) PCT Filed: Mar. 27, 2013
(86) PCT No.: PCT/EP2013/056627

§ 371 Date: Jun. 23, 2015

(30) Foreign Application Priority Data
Mar. 27, 2012 (EP) 12161628.8
Sep. 3, 2012 (EP) 12182857.8

(51) Int. Cl. G01N 33/68

(52) U.S. Cl. CPC G01N 33/6893; G01N 2800/102; G01N 2570/00; G01N 2800/60

(57) ABSTRACT

The present invention relates to a novel method for identifying marker sequences for rheumatoid arthritis, the novel marker sequences discovered with the aid of the method, and the diagnostic use thereof. The invention also relates to diagnostic devices containing such marker sequences for rheumatoid arthritis, in particular a protein biochip or beads (pellets), and use thereof.

The present invention relates to a novel method for identifying marker sequences for rheumatoid arthritis, the novel marker sequences discovered with the aid of the method, and diagnostic use thereof. The invention also relates to diagnostic devices containing such marker sequences for rheumatoid arthritis, in particular a protein biochip or beads, and use thereof.

Protein biochips are gaining increasing industrial importance in analysis and diagnosis as well as in pharmaceutical development. Protein biochips have become established as screening tools.

Here, the rapid and highly parallel detection of a multiplicity of specifically binding analysis molecules in a single experiment is made possible. To produce protein biochips, it is necessary to have the required proteins available. In particular, protein expression libraries have been established for this purpose. High-throughput cloning of defined open reading frames is one possibility (Heyman, J. A., Cornthwaite, J., Foncerrada, L., Gilmore, J. R., Gontang, E., Hartman, K. J., Hernandez, C. L., Hood, R., Hull, H. M., Lee, W. Y., Marcil, R., Marsh, E. J., Mudd, K. M., Patino, M. J., Purcell, T. J., Rowland, J. J., Sindici, M. L. and Hoeffler, J. P. (1999) Genome-scale cloning and expression of individual open reading frames using topoisomerase I-mediated ligation. Genome Res, 9, 383-392; Kersten, B., Feilner, T., Kramer, A., Wehrmeyer, S., Possling, A., Witt, I., Zanor, M. I., Stracke, R., Lueking, A., Kreutzberger, J., Lehrach, H. and Cahill, D. J. (2003) Generation of Arabidopsis protein chip for antibody and serum screening. Plant Molecular Biology, 52, 999-1010; Reboul, J., Vaglio, P., Rual, J. F., Lamesch, P., Martinez, M., Armstrong, C. M., Li, S., Jacotot, L., Bertin, N., Janky, R., Moore, T., Hudson, J. R., Jr., Hartley, J. L., Brasch, M. A., Vandenhaute, J., Boulton, S., Endress, G. A., Jenna, S., Chevet, E., Papasotiropoulos, V., Tolias, P. P., Ptacek, J., Snyder, M., Huang, R., Chance, M. R., Lee, H., Doucette-Stamm, L., Hill, D. E. and Vidal, M. (2003) C. elegans ORFeome version 1.1: experimental verification of the genome annotation and resource for proteome-scale protein expression. Nat Genet, 34, 35-41.; Walhout, A. J., Temple, G. F., Brasch, M. A., Hartley, J. L., Lorson, M. A., van den Heuvel, S. and Vidal, M. (2000) GATEWAY recombinational cloning: application to the cloning of large numbers of open reading frames or ORFeomes. Methods Enzymol, 328, 575-592). However, such an approach is closely linked to the progress of the genome sequencing projects and the annotation of these gene sequences. In addition, the determination of the expressed sequence is not always clear due to differential splicing processes. This problem can be avoided by the use of cDNA expression libraries (Büssow, K., Cahill, D., Nietfeld, W., Bancroft, D., Scherzinger, E., Lehrach, H. and Walter, G. (1998) A method for global protein expression and antibody screening on high-density filters of an arrayed cDNA library. Nucleic Acids Research, 26, 5007-5008; Büssow, K., Nordhoff, E., Lübbert, C., Lehrach, H. and Walter, G. (2000) A human cDNA library for high-throughput protein expression screening. Genomics, 65, 1-8; Holz, C., Lueking, A., Bovekamp, L., Gutjahr, C., Bolotina, N., Lehrach, H. and Cahill, D. J. (2001) A human cDNA expression library in yeast enriched for open reading frames. Genome Res, 11, 1730-1735; Lueking, A., Holz, C., Gotthold, C., Lehrach, H. and Cahill, D. (2000) A system for dual protein expression in Pichia pastoris and Escherichia coli, Protein Expr. Purif., 20, 372-378). Here, the cDNA of a specific tissue is cloned into a bacterial or eukaryotic expression vector, such as yeast. The vectors used for the expression are generally characterised in that they carry inducible promoters that may be used to control the time of protein expression. In addition, expression vectors have sequences for what are known as affinity epitopes or affinity proteins, which on the one hand permit the specific detection of the recombinant fusion proteins by means of an antibody directed against the affinity epitope, and on the other hand render possible the specific purification via affinity chromatography (IMAC).

By way of example, the gene products of a cDNA expression library from human foetal brain tissue in the bacterial expression system Escherichia coli were arranged in high-density format on a membrane and could be successfully screened with different antibodies. It was possible to show that the proportion of full-length proteins is at least 66%. Additionally, the recombinant proteins from expression libraries could be expressed and purified in a high-throughput manner (Braun P., Hu, Y., Shen, B., Halleck, A., Koundinya, M., Harlow, E. and LaBaer, J. (2002) Proteome-scale purification of human proteins from bacteria. Proc Natl Acad Sci USA, 99, 2654-2659; Büssow (2000) supra; Lueking, A., Horn, M., Eickhoff, H., Büssow, K., Lehrach, H. and Walter, G. (1999) Protein microarrays for gene expression and antibody screening. Analytical Biochemistry, 270, 103-111). Such protein biochips based on cDNA expression libraries are disclosed in particular in WO 99/57311 and WO 99/57312. Furthermore, in addition to antigen-presenting protein biochips, antibody-presenting arrangements are likewise described (Lal et al (2002) Antibody arrays: An embryonic but rapidly growing technology, DDT, 7, 143-149; Kusnezow et al. (2003), Antibody microarrays: An evaluation of production parameters, Proteomics, 3, 254-264).

Auger et al. (2009) Annals of the Rheumatic Diseases, British Medical Association, London, GB, vol. 68, Nr. 4, S. 591-594 discloses a method for identifying IgG autoantibodies in sera of patients with rheumatoid arthritis (RA). Here, serum samples of patients with RA are examined comparatively with those of healthy control individuals on the Invitrogen ProtoArray (8268 human proteins as GST fusion proteins, purified under native conditions and spotted onto a glass slide coated with nitrocellulose). The arrays are incubated with the serum samples and examined by Alexa Fluor 647 conjugated to anti-human IgG. A panel of measured values is evaluated by Z-score, CIP (Chebyshev Inequality Precision) and CV (Coefficient of Variation). In Auger et al. the antigens peptidylarginine deiminase 4 (PAD4), protein kinase Cβ1 (PKCβ1), phosphatidylinositol-4-phosphate-5-kinase type II γ (PIP4K2C) and v raf murine sarcoma viral oncogene homologue B1 catalytic domain (BRAF) were identified using this method. Auger et al. does not disclose the diagnostic use of the identified antigens.

EP 1 731 608 A1 discloses a method for identifying “genes susceptible to RA” by gene mapping with the aid of microsatellite markers and PCR techniques. With the aid of the method the genes TNXB, NOTCH4 (chromosome 6), RAB6A, MPRL48, FLJ11848, UCP2 and UCP3 (chromosome 11) were discovered in human genomic DNA. EP 1 731 608 A1 claims a marker gene for an RA test consisting of a partial DNA sequence of one of the discovered marker genes and comprising at least one SNP in human genomic DNA. In addition, a method is disclosed for detecting RA comprising the steps of obtaining partial DNA sequences corresponding to one of the marker genes from a subject to be examined, determining the nucleotide sequence of the partial DNA sequence, and comparing the nucleotide sequence to the corresponding nucleotide sequence obtained from a normal individual. A test kit for RA comprising one of the marker genes or a primer derived therefrom, a polypeptide coded by one of the marker genes, and a screening method is also disclosed.

WO 2009/138408 A2 claims a diagnostic method, in which an autoantigen marker comprising the catalytic domains of BRAF or an antibody fragment thereof is used to detect RA, wherein, where appropriate, anti-PAD4 antibodies are also detected in the biological sample of the subject to be examined. WO 2009/138408 A2 also discloses a detection kit for detecting anti-BRAF autoantibodies, an array with autoantigen markers comprising BRAF and PAD4 for diagnosing RA, and the use of an autoantigen marker comprising BRAF for diagnosing RA, preferably in patients who are CCP negative (page 3, paragraph

In WO 2009/138408 A2 the biomarkers were identified in that serum from RA patients and controls (patients with spondylarthropathy (AS)), systemic lupus erythematosus (SLE), systemic sclerosis (SSC) and healthy individuals) were screened with the ProtoArray Human Protein Microarray (Invitrogen), wherein the detection was performed by means of anti-human IgG conjugated to Alex Fluor 647. A panel of measured values was evaluated by Z-score, CIP and CV.

WO 2007/039280 A1 claims a method for the differential diagnosis of RA by determining the concentration of anti-CCP and anti-nuclear antibodies in a sample and correlation with the diagnosis of RA. Here, the markers CRP, SAA, IL-6, 5100, osteopontin, RF, MMP-1, MMP-3, hyaluronic acid, sCD14, angiogenesis markers and products from the metabolism of bone, cartilage or synovial membrane can be used in addition. WO 2007/039280 A1 also claims the use of a panel comprising anti-CCP and ANA for the diagnosis of RA, and a test kit.

WO 2005/032328 A2 claims a method for detecting RA in a sample of a patient, wherein the amount of one or more markers selected from Table 1 (679 are specified there) or Table 2 (some of the markers from Table 1) compared to a control with normal expression level of the respective marker is determined. WO 2005/032328 A2 also discloses distinguishing between progressive and non-progressive RA with certain markers.

In WO 2005/032328 A2 the markers for RA are identified in the serum of patients by means of MS.

WO 2005/061692 A1 claims a protein microarray comprising at least two of the proteins selected from L35 protein, eukaryotic translation elongation factor 1 α. 2, NADH dehydrogenase 3 (complex I), 24-kDa sub-unit of complex I, mitotic kinesin-like protein-1, thromboxane synthase and uncoupling protein homologue, wherein the proteins are HIS-tagged and printed onto a glass slide coated with Ni²⁺. WO 2005/061692 A1 also specifies a method for screening RA with use of the specified proteins, a drug containing one of the proteins, and a kit for screening RA. In order to identify the above-mentioned proteins, it is proposed in WO 2005/061692 A1 to compare the serum of afflicted patients with that of healthy control individuals (page 6, paragraph 3).

Nicaise et al. (2008) Arthritis Research Therapy, Biomed Central LTD, GB, vol. 10, Nr. 6, S. R142-R142.7 examines the suitability of anti-MCV (mutated citrullinated vimentin) antibodies for the diagnosis of RA in CCP-negative patients and the use for monitoring during therapy with Infliximab. Here, groups of patients with RA and CCP and with RA and without CCP are compared with patients having other rheumatic diseases (psoriatic rheumatism, primary Sjögren's syndrome, ankylosis spondylitis) and healthy control individuals. The use of an array/an arrangement is not described.

Vossenaar et al. (2004) Clinical and Applied Immunology Reviews 4, 239-262 concerns the use of citrullinated autoantigens and of anti-CCP antibodies and antigens thereof as serological markers for the detection of RA. Vossenaar et al. proposes using microarray technology to analyse autoantibody profiles of RA patients (page 254, paragraph 2).

US 2007/0254300 A1 uses a yeast two-hybrid system, in order to identify anti-inflammatory compounds ([0504] to [0506]) and claims a protein complex, wherein the first protein is PAK, a fragment thereof, or a fusion protein containing this, and the second protein is ERK3, PRKAR1A, KRT23(209), PN7098, AL117237, PCNT2, PROX1, HOOK1, IGHG1, GOLGA2, KIAA0555, LRPPRC or a fragment of these proteins. US 2007/0254300 A1 also discloses a microarray comprising this protein complex and a method for discovering “modulators” of the protein complex using this microarray. A method for detecting a change in an inflammatory disease, for example RA, is also claimed, wherein the sample of a patient is examined to ascertain whether a change in the expression level of one of the proteins in Tables 1 to 82 (82 different proteins are specified here) or in the nucleotide sequence of a gene coding for the 82 proteins is determined compared with patients without this inflammatory disease.

WO 2009/030226 discloses marker sequences for rheumatoid arthritis and diagnostic use thereof as well as a method for screening potential active ingredients for rheumatoid arthritis by means of these marker sequences. A diagnostic device containing such marker sequences for rheumatoid arthritis, in particular a protein biochip, and use thereof are also disclosed.

DE 10 2007 041 656 A1 discloses the use of marker sequences for diagnosing RA, methods for diagnosing RA with use of these marker sequences, methods for stratification, an arrangement of marker sequences, an assay/protein biochip, the use of the arrangement, diagnostic agents comprising the marker sequences, a target for treatment and therapy, and the use of the marker sequences to carry out an apheresis.

The RA-specific expression clones were obtained in DE 10 2007 041 656 A1 by screening 10 or more patient samples individually against a cDNA expression library and were identified by comparison with 10 or more healthy samples. FIG. 1 shows the differential screening between two protein biochips, one from a cDNA expression bank of a patient and one from a healthy test subject. The differential clones are detected by means of fluorescence labelling and evaluated by means of bioinformatics.

There is still a pressing need for indication-specific diagnostic devices for rheumatoid arthritis.

The object of the present invention is therefore to discover improved marker sequences for rheumatoid arthritis and to specify a diagnostic use thereof.

The provision of specific marker sequences allows a reliable diagnosis and stratification of patients with rheumatoid arthritis.

In a two-stage method comprising firstly the selection of marker sequence candidates with the aid of protein biochips and the subsequent validation thereof by means of beads, highly specific marker sequences are discovered for rheumatoid arthritis.

The invention relates to a method for identifying marker sequences for rheumatoid arthritis (RA), comprising the steps of:
a) selecting marker sequence candidates by screening protein biochips representing a cDNA expression library with at least 10 patient samples and at least 10 samples of healthy individuals, wherein each sample is measured individually and marker sequence candidates for RA are selected by a comparison of the results of the screens obtained with the RA patient samples and the results of the screens obtained with the samples of healthy individuals,
b) producing the proteins and/or partial proteins (peptides) coded by the marker sequence candidates by expression of the cDNA of the marker sequence candidates,
c) producing beads to which one or more of the proteins and/or partial proteins (peptides) produced in step b) are coupled,
d) validating the marker sequence candidates coupled to the beads by means of samples from patients with RA and samples from healthy individuals in that marker sequences for RA demonstrate an interaction with the samples from patients with RA and demonstrate a comparatively lower or no interaction with the samples from healthy individuals, wherein the marker sequences SEQ ID No. 1 to 182, SEQ ID. No. 183 to 273, SEQ ID No. 274 to 313 and SEQ ID No. 314 to SEQ ID No. 333 are obtained.

In the field of microarrays flat substrates are used, to which marker sequences or sequences to be examined are bound. In protein biochips the marker sequences to be examined or the sequences binding to these marker sequences are immobilised on a solid, flat support. An alternative arrangement of marker sequences or sequences to be examined is possible on beads, which therefore differ inter alia in view of their sensitivity and specificity from conventional microarrays. Bead arrays are created for example by impregnating pellets either with different concentrations of fluorescent dye or for example by barcode technology. The pellets can be addressed and can be used to identify specific binding events that occur on their surface. Bead technology is based on microscopically small spherical pellets or platelets, which are referred to as microspheres or beads. These beads can serve analogously to ELISA and Western Blot as solid phase for biochemical detection reactions. A wide range of different bead types are available, which for example differ in their fluorescence shade and each of which carries its own specific detection reagent on the surface. In this way, an accordingly large number of different detection reactions can be carried out simultaneously in a very small sample volume. With bead arrays specific interactions between two defined biochemical compounds can be detected. Compared with conventional microarrays, the bead-based validation is characterised by a particularly high sensitivity and specificity. With the two-stage method according to the invention and the use of beads for validation, marker sequences for RA can be identified that differ in terms of their sensitivity and specificity from the previously known marker sequences. The two-stage method according to the invention has not been described previously in the prior art. In the method according to the invention other biomarkers can additionally be coupled to the beads. Marker sequences with specific specificity can thus be obtained. By way of example, marker sequences with which sub-groups of patients within the indication RA can be diagnosed are obtained.

In one embodiment of the method steps c) and d) are performed in the presence of CCP (cytochrome c peroxidase). The marker sequences validated in the presence of CCP are more sensitive with respect to the diagnosis of rheumatoid arthritis than CCP. With the aid of the marker sequences validated in the presence of CCP, a sub-group of RA patients can be diagnosed. When performing the validation in the presence of CCP, the marker sequences SEQ ID No. 29 to 79, SEQ ID No. 120 to 170, SEQ ID No. 211 to 261, SEQ ID No. 274, SEQ ID No. 276, SEQ ID No. 278, SEQ ID No. 285 to 288, SEQ ID No. 291, SEQ ID No. 294, SEQ ID No. 296, SEQ ID No. 298, SEQ ID No. 305 to 308, SEQ ID No. 311, SEQ ID No. 314, SEQ ID No. 316, SEQ ID No. 318, SEQ ID No. 325 to 328, and SEQ ID No. 331 are obtained.

The invention relates to a method for identifying marker sequences for rheumatoid arthritis (RA) comprising the steps of:
a) selecting marker sequence candidates by screening protein biochips representing a cDNA expression library with at least 10 patient samples and at least 10 samples of healthy individuals, wherein each sample is measured individually and marker sequence candidates for RA are selected by a comparison of the results of the screens obtained with the RA patient samples and the results of the screens obtained with the samples of healthy individuals,
b) producing the proteins and/or partial proteins (peptides) coded by the marker sequence candidates by expression of the cDNA of the marker sequence candidates,
c) producing beads to which one or more of the proteins and/or partial proteins (peptides) produced in step b) are coupled, wherein CCP (cytochrome c peroxidase) is also coupled to the beads,
d) validating the marker sequence candidates coupled to the beads from c) by means of samples from patients with RA and samples from healthy individuals in that marker sequences for RA demonstrate an interaction with the samples from patients with RA and demonstrate a comparatively lower or no interaction with the samples from healthy individuals, wherein the marker sequences SEQ ID No. 29 to 79, SEQ ID No. 120 to 170, SEQ ID No. 211 to 261, SEQ ID No. 274, SEQ ID No. 276, SEQ ID No. 278, SEQ ID No. 285 to 288, SEQ ID No. 291, SEQ ID No. 294, SEQ ID No. 296, SEQ ID No. 298, SEQ ID No. 305 to 308, SEQ ID No. 311, SEQ ID No. 314, SEQ ID No. 316, SEQ ID No. 318, SEQ ID No. 325 to 328, and SEQ ID No. 331 are obtained.

One embodiment of the method according to the invention is characterised in that an interaction between marker sequence candidates and the samples in method step d) is detected by means of a fluorescence signal, wherein the intensity of the fluorescence signal correlates with the intensity of the interaction.

The invention also relates to a marker sequence for rheumatoid arthritis obtainable by a method according to the invention, wherein the marker sequence is selected from the group of sequences SEQ ID No. 1 to 182 and SEQ ID. No. 274 to 313, a sequence homologous to the sequences SEQ ID No. 1 to 182 or SEQ ID No. 274 to 313 or a partial sequence of SEQ ID No. 1 to 182 or SEQ ID. No. 274 to 313 or a protein coded by SEQ ID No. 1 to 182 or SEQ ID. No. 274 to 313 or a protein coded by a partial sequence of SEQ ID No. 1 to 182 or SEQ ID. No. 274 to 313 or a protein coded by a homologous sequence of SEQ ID No. 1 to 182 or SEQ ID. No. 274 to 313 and a genomic sequence comprising one of the sequences SEQ ID No. 1 to 182 or SEQ ID. No. 274 to 313.

The invention also relates to a marker sequence for rheumatoid arthritis in CCP-negative patients obtainable by a method according to the invention, wherein the marker sequence is selected from the group of sequences SEQ ID No. 29 to 79, SEQ ID No. 274, SEQ ID No. 276, SEQ ID No. 278, SEQ ID No. 285 to 288, SEQ ID No. 291 or SEQ ID. No. 120 to 170, SEQ ID No. 294, SEQ ID No. 296, SEQ ID No. 298, SEQ ID No. 305 to 308, SEQ ID No. 311, a sequence homologous to the sequences SEQ ID No. 29 to 79, SEQ ID No. 274, SEQ ID No. 276, SEQ ID No. 278, SEQ ID No. 285 to 288, SEQ ID No. 291 or SEQ ID No. 120 to 170, SEQ ID No. 294, SEQ ID No. 296, SEQ ID No. 298, SEQ ID No. 305 to 308, SEQ ID No. 311 or a partial sequence of SEQ ID No. 29 to 79, SEQ ID No. 274, SEQ ID No. 276, SEQ ID No. 278, SEQ ID No. 285 to 288, SEQ ID No. 291 or SEQ ID. No. 120 to 170, SEQ ID No. 294, SEQ ID No. 296, SEQ ID No. 298, SEQ ID No. 305 to 308, SEQ ID No. 311 or a protein coded by SEQ ID No. 29 to 79, SEQ ID No. 274, SEQ ID No. 276, SEQ ID No. 278, SEQ ID No. 285 to 288, SEQ ID No. 291 or SEQ ID. No. 120 to 170, SEQ ID No. 294, SEQ ID No. 296, SEQ ID No. 298, SEQ ID No. 305 to 308, SEQ ID No. 311 or a protein coded by a partial sequence of SEQ ID No. 29 to 79, SEQ ID No. 274, SEQ ID No. 276, SEQ ID No. 278, SEQ ID No. 285 to 288, SEQ ID No. 291 or SEQ ID. No. 120 to 170, SEQ ID No. 294, SEQ ID No. 296, SEQ ID No. 298, SEQ ID No. 305 to 308, SEQ ID No. 311 or a protein coded by a homologous sequence of SEQ ID No. 29 to 79, SEQ ID No. 274,

SEQ ID No. 276, SEQ ID No. 278, SEQ ID No. 285 to 288, SEQ ID No. 291 or SEQ ID. No. 120 to 170, SEQ ID No. 294, SEQ ID No. 296, SEQ ID No. 298, SEQ ID No. 305 to 308, SEQ ID No. 311 or a genomic sequence comprising one of the sequences SEQ ID No. 29 to 79, SEQ ID No. 274, SEQ ID No. 276, SEQ ID No. 278, SEQ ID No. 285 to 288, SEQ ID No. 291 or SEQ ID. No. 120 to 170, SEQ ID No. 294, SEQ ID No. 296, SEQ ID No. 298, SEQ ID No. 305 to 308, or SEQ ID No. 311.

The invention also relates to a marker sequence for rheumatoid arthritis obtainable by a method according to the invention, wherein the marker sequence is selected from the group of sequences SEQ ID No. 183 to 273, SEQ ID No. 314 to 333, in particular SEQ ID No. 211 to 261, SEQ ID No. 314, SEQ ID No. 316, SEQ ID No. 318, SEQ ID No. 325 to 328, and SEQ ID No. 331.

The invention also relates to the use of one or more marker sequence(s) according to the invention for the diagnosis of rheumatoid arthritis.

One embodiment concerns the use according to the invention, wherein the marker sequence(s) is/are determined on or from a patient to be examined.

One embodiment concerns the use according to the invention, characterised in that 2 or 3, preferably 4 or 5, particularly preferably 6, 7 or 8 or more different marker sequences, for example 10 to 20 or 30 or more different marker sequences, are determined on or from a patient to be examined.

One embodiment concerns the use according to the invention, characterised in that the marker sequence(s) is/are applied to a solid support, wherein the solid support is selected from filters, membranes, wafers, for example silicon wafers, glass, metal, plastic, chips, mass spectrometry targets, matrices, and beads, for example magnetic, coated or labelled beads, such as fluorophore-labelled beads or Luminex beads.

The invention also relates to a method for diagnosing rheumatoid arthritis, wherein

a.) at least one marker sequence according to the invention is applied to a solid support, preferably to a bead and
b.) is brought into contact with bodily fluid or tissue sample of a patient and
c.) an interaction of the bodily fluid or of the tissue sample with the marker sequence from a.) is detected.

Such an interaction can be detected for example by a probe, in particular by an antibody.

The invention also relates to a method for stratification, in particular for risk stratification, or for therapy management of a patient with rheumatoid arthritis, wherein at least one marker sequence according to the invention is used in order to examine a sample from the patient.

One embodiment concerns a method according to the invention for diagnosing rheumatoid arthritis, wherein the stratification or the therapy management includes decisions regarding the treatment and therapy of the patient, in particular the hospitalisation of the patient, the use, efficacy and/or dosage of one or more drugs, a therapeutic measure or the monitoring of the course of a disease and the course of therapy, aetiology or classification of a disease, inclusive of prognosis.

The invention also relates to an arrangement comprising or consisting of one or more marker sequence(s) according to the invention.

The invention also relates to an assay or protein array comprising an arrangement according to the invention.

The invention also relates to the use of an arrangement according to the invention or of an assay or protein array according to the invention for identifying and/or characterising a substance for rheumatoid arthritis containing means for detecting binding success, characterised in that an arrangement or an assay or protein array is brought into contact with a.) at least one substance to be examined and b.) binding success is detected.

The invention also relates to a diagnostic agent for the diagnosis of rheumatoid arthritis containing at least one marker sequence according to the invention and where appropriate further auxiliaries and additives.

The invention also relates to a target for the treatment or therapy of rheumatoid arthritis, wherein the target is selected from the marker sequences according to the invention.

The invention also relates to the use of one or more marker sequence(s) according to the invention as affinity material for carrying out an apheresis or blood washing for patients with rheumatoid arthritis. Here, an arrangement comprising one or more marker sequences according to the invention is preferably used as affinity material for carrying out the apheresis or blood washing, wherein substances from bodily fluids from a patient with rheumatoid arthritis, such as blood or plasma, bind to the marker sequences according to the invention and consequently can be removed selectively from the bodily fluid. Corresponding devices are known accordingly, such as chromatography devices containing beads, balls or chromatographic material, for example in a column, which comprise the marker sequences according to the invention and therefore can remove (auto)antibodies selectively, for example.

The invention also relates to the use of at least one marker sequence according to the invention for identifying a sub-group of patients within the group of patients with rheumatoid arthritis, wherein the patients in the sub-group cannot be identified by means of the marker CCP and/or cannot be identified with the markers known in the prior art or marker sequences for rheumatoid arthritis.

The invention therefore relates to the use of marker sequences for the diagnosis of rheumatoid arthritis, wherein at least one marker sequence selected from the group of marker sequences SEQ ID No. 1 to 91 or SEQ ID No. 274 to 293 and/or SEQ ID No. 92 to 182 or SEQ ID No. 294 to 313 and/or the genomic sequences comprising one of the sequences SEQ ID No. 1 to 182 or SEQ ID No. 274 to 313 and/or a protein coded by the sequences SEQ ID No. 1 to 182 or SEQ ID. No. 274 to 313 or a partial sequence or a homologue of the sequences SEQ ID No. 1 to 182 or SEQ ID No. 274 to 313 or a protein coded by the partial sequence or the homologous sequence is determined on or from a patient to be examined, and wherein the marker sequence(s) is/are identified by a method according to the invention.

A particular embodiment of the method according to the invention comprises the steps of
a) identifying marker sequence candidates by differential screening with protein biochips from a cDNA expression bank of a patient with rheumatoid arthritis and from a test subject without rheumatoid arthritis,
b) expressing the marker sequence candidates for production of the coded proteins and/or partial proteins (peptides),
c) producing Luminex beads, to which one or more of the proteins and/or partial proteins (peptides) produced in step b) are coupled, wherein, where appropriate, biomarkers already known for rheumatoid arthritis, for example CCP (cytochrome c peroxidase), are coupled to the Luminex beads,
d) validating the marker sequence candidates by means of samples from patients with rheumatoid arthritis and test subjects without rheumatoid arthritis, wherein the validation is performed, where appropriate, in the presence of the biomarkers already known for rheumatoid arthritis, for example CCP.

A preferred embodiment of the invention concerns the use of marker sequences according to the invention for the diagnosis of rheumatoid arthritis in patients that are CCP negative, wherein at least one marker sequence selected from the group of marker sequences SEQ ID No. 29 to 79 and/or SEQ ID No. 120 to 170 and/or the genomic sequences comprising one of the sequences SEQ ID No. 29 to 79 or 120 to 170 and/or a protein coded by the sequences SEQ ID No. 29 to 79 or 120 to 170 or a partial sequence or a homologue of the sequences SEQ ID No. 29 to 79 or SEQ ID No. 120 to 170 or a protein coded by the partial sequence or the homologue sequence is determined on or from a patient to be examined.

The marker sequences validated in the presence of CCP are more sensitive with respect to the diagnosis of rheumatoid arthritis than CCP. A sub-group of patients that cannot be identified with the aid of the marker CCP already known for rheumatoid arthritis can be identified and/or monitored in this way. With the aid of these marker sequences, rheumatoid arthritis can also be diagnosed in an earlier stage than with CCP.

A further particular embodiment of the invention relates to the use of marker sequences according to the invention for the diagnosis of rheumatoid arthritis at an early stage of RA, wherein at this early stage RA cannot yet be detected by means of CCP, and wherein at least one marker sequence selected from the group of marker sequences SEQ ID No. 29 to 79 and/or SEQ ID No. 120 to 170 and/or the genomic sequences comprising one of the sequences SEQ ID No. 29 to 79 or 120 to 170 and/or a protein coded by the sequences SEQ ID No. 29 to 79 or 120 to 170 or a partial sequence or a homologue of the sequences SEQ ID No. 29 to 79 or SEQ ID No. 120 to 170 or a protein coded by the partial sequence or the homologous sequence is determined on or from a patient to be examined.

A further embodiment of the invention concerns the use of the marker sequence(s) according to the invention for the diagnosis of rheumatoid arthritis, characterised in that the determination is performed by means of in-vitro diagnosis.

A further embodiment of the invention concerns diagnostic agents for the diagnosis of rheumatoid arthritis containing at least one marker sequence selected from the group of marker sequences SEQ ID No. 1 to 91 or SEQ ID No. 274 to 293 and/or SEQ ID No. 92 to 182 or SEQ ID No. 294 to 313 and/or the genomic sequences comprising one of the sequences SEQ ID No. 1 to 182 or SEQ ID No. 274 to 313 and/or a protein coded by the sequences SEQ ID No. 1 to 182 or SEQ ID. No. 274 to 313 or a partial sequence or a homologue of the sequences SEQ ID No. 1 to 182 or SEQ ID No. 274 to 313 or a protein coded by the partial sequence or the homologous sequence, wherein the marker sequence(s) was/were identified using a method comprising the steps of
a) selecting marker sequence candidates by screening protein biochips representing a cDNA expression library with at least 10 patient samples and at least 10 samples of healthy individuals, wherein each sample is measured individually and marker sequence candidates for RA are selected by a comparison of the results of the screens obtained with the RA patient samples and the results of the screens obtained with the samples of healthy individuals,
b) producing the proteins and/or partial proteins (peptides) coded by the marker sequence candidates by expression of the cDNA of the marker sequence candidates,
c) producing beads to which one or more of the proteins and/or partial proteins (peptides) produced in step b) and where appropriate CCP are coupled,
d) validating the marker sequence candidates coupled to the beads by means of samples from patients with RA and samples from healthy individuals in that marker sequences for RA demonstrate an interaction with the samples from patients with RA and demonstrate a comparatively lower or no interaction with the samples from healthy individuals, wherein the marker sequences SEQ ID No. 1 to 182, SEQ ID. No. 183 to 273, SEQ ID No. 274 to 313 and SEQ ID No. 314 to SEQ ID No. 333, or in the presence of CCP the marker sequences SEQ ID No. 29 to 79, SEQ ID No. 120 to 170, SEQ ID No. 211 to 261, SEQ ID No. 274, SEQ ID No. 276, SEQ ID No. 278, SEQ ID No. 285 to 288, SEQ ID No. 291, SEQ ID No. 294, SEQ ID No. 296, SEQ ID No. 298, SEQ ID No. 305 to 308, SEQ ID No. 311, SEQ ID No. 314, SEQ ID No. 316, SEQ ID No. 318, SEQ ID No. 325 to 328, and SEQ ID No. 331 are obtained.

The marker sequences according to the invention were able to be identified by means of differential screening of samples from healthy test subjects with patient samples with rheumatoid arthritis. The marker sequences according to the invention were then expressed and, following coupling of the expressed marker sequence candidates to Luminex beads, validated with the aid of the Luminex beads, partly by comparison with known biomarkers for rheumatoid arthritis and as described in the practical examples. Highly specific marker sequences could thus be identified for rheumatoid arthritis.

“Beads” (pearls, pellets, originally also referred to as latex particles) designate what are known as microspheres or microparticles, which are used as supports for biomolecules in tests and assays. Uniform (approximately equally sized) microparticles that are produced by special chemical methods are required for tests and assays. These methods are known to a person skilled in the art. Beads for different applications are also commercially available (for example from the company Progen Biotechnik GmbH). Beads may consist of different materials, for example glass, polystyrene, PMMA and different other polymers, partly also copolymers. Beads can be labelled with different dyes or dye mixtures and can be provided with coatings. Biomolecules can be coupled to the surface of beads. Different coupling methods are available for this purpose and are known to a person skilled in the art, for example adsorption or covalent coupling. The surface of the beads can be modified, such that a directed coupling of the biomolecules on the bead surface, for example in conjunction with spacers, tags or special modifications, is possible, and whereby the analytical sensitivity can be further increased.

The term “rheumatoid arthritis (RA)” is defined for example by Pschyrembel, de Gruyter, 261^{st} edition (2007), Berlin. In accordance with the invention “juvenile idiopathic arthritis” (ICD-10: M08.-. abb: JIA. earlier synonyms: juvenile rheumatoid arthritis, juvenile chronic arthritis, Still's disease or popular name: child's rheumatism) is the collective term for a series of diseases primarily affecting the joints (arthritis) of rheumatic origin in childhood (juvenile) (definition for example according to Pschyrembel, de Gruyter, 261^{st} edition (2007), Berlin). This is a polygenic disease that can be diagnosed particularly advantageously by means of the marker sequences according to the invention, preferably SEQ ID No. 1 to 333.

In a further embodiment at least 2 to 5 or 10, preferably 30 to 50 marker sequences or 50 to 100 or more marker sequences are determined on or from a patient to be examined.

In a further embodiment of the invention the marker sequences according to the invention can also be combined, supplemented, consolidated or expanded with known biomarkers for this indication.

In a preferred embodiment the marker sequences are determined outside the human body and the determination is performed in an ex vivo/in vitro diagnosis.

A further object of the invention is therefore also to provide a diagnostic device or an assay, in particular a protein biochip, that allows a diagnosis or examination for rheumatoid arthritis.

In the sense of this invention, “diagnosis” means the positive determination of rheumatoid arthritis by means of the marker sequences according to the invention as well as the assignment of the patients to the indication rheumatoid arthritis. The term diagnosis includes the medical diagnostics and examinations in this regard, in particular in-vitro diagnostics and laboratory diagnostics, and also proteomics and nucleic acid blotting. Further tests may be necessary to be sure and to exclude other diseases. The term diagnosis therefore also includes the differential diagnosis of rheumatoid arthritis by means of the marker sequences according to the invention, and the prognosis in the case of determined rheumatoid arthritis.

The invention also relates to a method for the stratification, in particular risk stratification and/or therapy management of a patient with rheumatoid arthritis, for example in a patient with a very early stage of RA or RA that cannot be detected by means of the marker CCP, wherein at least one marker sequence according to the invention is determined on a patient to be examined. The stratification of the patient with rheumatoid arthritis in new or established sub-groups within the disease rheumatoid arthritis is also included, as well as the expedient selection of patient groups for the clinical development of new therapeutic agents or the selection for therapy with certain active agents. The term therapy management also includes the division of patients into responders and non-responders in respect of a therapy or the course of a therapy.

In the sense of this invention, “stratification or therapy management” means that the method according to the invention renders possible decisions for the treatment and therapy of the patient, whether it is the hospitalisation of the patient, the use, efficacy and/or dosage of one or more drugs, a therapeutic measure, or the monitoring of the course of a disease and the course of therapy or aetiology or classification of RA, for example into a new or existing sub-type, or the differentiation of RA and patients thereof.

In a further embodiment of the invention, the term “stratification” in particular includes the risk stratification with the prognosis of an “outcome” of a negative health event.

Within the scope of this invention, the term “patient” is understood to mean any test subject (human or mammal), with the provision that the test subject is tested for rheumatoid arthritis.

The term “marker sequences” in the sense of this invention means that the nucleic acid sequence, for example the mRNA, cDNA or the polypeptide or protein obtainable therefrom are significant for rheumatoid arthritis. By way of example the mRNA or cDNA or the polypeptide or protein obtainable therefrom can interact with substances from the bodily fluid or tissue sample of a patient with rheumatoid arthritis (for example antigen (epitope)/antibody (paratope) interaction).

In the sense of the invention “wherein at least one marker sequence selected from the group of marker sequences SEQ ID No. 1 to 182 or SEQ ID No. 274 to 313, the genomic sequences comprising one of the sequences SEQ ID No. 1 to 182 or SEQ ID No. 274 to 313, or a protein coded by the sequences SEQ ID No. 1 to 182 or SEQ ID. No. 274 to 313 or a partial sequence or a homologue of the sequences SEQ ID No. 1 to 182 or SEQ ID No. 274 to 313 or a protein coded by the partial sequence or the homologous sequence is determined on or from a patient to be examined” means that an interaction between the bodily fluid or the tissue sample of a patient and the marker sequence(s) according to the invention is detected. Such an interaction is, for example, a binding, in particular a binding substance at least at one of the marker sequences according to the invention, or in the case of a cDNA is the hybridisation with a suitable substance under selected conditions, in particular stringent conditions (for example as defined typically in J. Sambrook, E. F. Fritsch, T. Maniatis (1989), Molecular cloning: A laboratory manual, 2nd Edition, Cold Spring Harbor Laboratory Press, Cold Spring Habor, USA or Ausubel, “Current Protocols in Molecular Biology”, Green Publishing Associates and Wiley Interscience, N.Y. (1989)). One example for stringent hybridisation conditions is: hybridisation in 4×SSC at 65° C. (alternatively in 50% formamide and 4×SSC at 42° C.), followed by a number of washing steps in 0.1×SSC at 65° C. for a total of about one hour. One example for less stringent hybridisation conditions is hybridisation in 4×SSC at 37° C., followed by a number of washing steps in 1×SSC at room temperature.

Such substances, in accordance with the invention, are part of a bodily fluid, in particular blood, whole blood, blood plasma, blood serum, patient serum, urine, cerebrospinal fluid, synovial fluid, or a tissue sample of the patient.

In a further embodiment of the invention the marker sequences according to the invention can be present in the examined test subjects in a significantly higher or lower expression rate or concentration compared with the expression rate or concentration of the marker sequence in question in a healthy individual or in a test subject without RA. The increased or reduced expression rate or concentration is an indication of rheumatoid arthritis and the diagnosis RA. The relative expression rates diseased/healthy of the marker sequences according to the invention can be determined for example by means of proteomics or nucleic acid blotting.

The marker sequences according to the invention, in a further embodiment of the invention, have an identification signal, which is addressed to the substance to be bound (for example antibody, nucleic acid). In accordance with the invention, the recognition signal for a protein is preferably an epitope and/or paratope and/or hapten, and for a cDNA is preferably a hybridisation or binding region. In a particularly preferred embodiment of the invention the marker sequences according to the invention identify autoantibodies that are specific for RA or that are formed and/or are formed to an increased or reduced degree with the onset and the development of the RA disease. Two or more marker sequences according to the invention can be used to detect autoantibody profiles or changes in autoantibody profiles during therapy or during the course of the disease or to monitor such changes within the scope of follow-up care.

The marker sequences according to the invention SEQ ID No. 1 to 91 and 274 to 293 are specified in Table 7 and can be unambiguously identified (see RefSeq Accession or GI Accession) by the respective cited database entries (also by means of Internet: http://www.ncbi.nlm.nih.gov/).

The invention therefore also relates to the full-length sequences of the marker sequences according to the invention and the marker sequences as defined in the tables via the known database entries and also the marker sequences specified in the accompanying sequence protocol.

The invention furthermore likewise includes analogous embodiments of the marker sequences, in particular of the nucleic acid sequences SEQ ID No. 1 to 182 and SEQ ID No. 274 to 313 and the protein sequences SEQ ID No. 183 to 273 and SEQ ID No. 314 to 333, in particular the nucleic acid sequences SEQ ID No. 29 to 79 and SEQ ID No. 120 to 170, SEQ ID No. 274, SEQ ID No. 276, SEQ ID No. 278, SEQ ID No. 285 to 288, SEQ ID No. 291, SEQ ID No. 294, SEQ ID No. 296, SEQ ID No. 298, SEQ ID No. 305 to 308, SEQ ID No. 311, and the protein sequences SEQ ID No. 211 to 261, SEQ ID No. 314, SEQ ID No. 316, SEQ ID No. 318, SEQ ID No. 325 to 328, SEQ ID No. 331 as presented in the claims for example. The clone sequences according to the invention SEQ ID No. 1 to 91 and SEQ ID No. 274 to 293 are partial sequences, at least with high homology, of the marker sequences according to the invention SEQ ID No. 92 to 182 and SEQ ID No. 294 to 313. The marker sequences SEQ ID No. 29 to 79, SEQ ID No. 274, SEQ ID No. 276, SEQ ID No. 278, SEQ ID No. 285 to 288, SEQ ID No. 291 and the proteins coded by these marker sequences are particularly preferred.

In a further embodiment of the invention marker sequences are preferred that have P-values less than or equal to 0.006, preferably less than or equal to 0.001 or less than or equal to 0.0001, particularly preferably less than or equal to 0.00001 (see FIG. 1, Tables 8 and 8a). In another embodiment of the invention the marker sequences SEQ ID No. SEQ ID No. 4 to 15, partial sequences and homologues of SEQ ID No. 4 to 15, and also the peptides/proteins coded thereby are preferred, since these marker sequences have particularly suitable P-values. The P-value specifies the likelihood with which a match has been found in the database. For example, see http://www.ncbi.nlm.nih.gov/books/NBK62051/for a definition of the P-value.

In a further embodiment of the invention homologues of the marker sequences according to the invention are included. In particular, these are homologues having an identity of 70%, 80% or 85%, preferably 90%, 91%, 92%, 93%, 94% or 95% identity, in particular 96%, 97%, 98%, 99% or more identity, with the marker sequences according to the invention and suitable for the use according to the invention—the detection of rheumatoid arthritis (“homologues” or homologous marker sequences). Homologues can be protein sequences or nucleic acid sequences.

Partial sequences are sequences that comprise 50 to 100 nucleotides or amino acids, preferably 70-120 nucleotides or amino acids, particularly preferably 100 to 200 nucleotides or amino acids of one of the marker sequences SEQ ID No. 1 to 333.

In accordance with the invention the marker sequences also comprise modifications of the nucleotide sequence, for example of the cDNA sequence and the corresponding amino acid sequence, such as chemical modification, such as citrullination, acetylation, phosphorylation, glycosylation or polyA strand and further modifications known accordingly to a person skilled in the art.

In a further embodiment the respective marker sequence can be represented in different amounts in one or more regions on a solid support, for example a bead. This allows a variation of the sensitivity. The regions may each comprise a totality of marker sequences, i.e. a sufficient number of different marker sequences, in particular 2 to 5 or 10 or more marker sequences, and where appropriate further nucleic acids and/or proteins, in particular biomarkers. However, at least 96 to 25,000 (numerically) or more different or identical marker sequences and further nucleic acids and/or proteins, in particular biomarkers, are preferred. Furthermore, more than 2,500 different or identical marker sequences are preferred, particularly preferably 10,000 or more, and where appropriate further nucleic acids and/or proteins, in particular biomarkers.

The invention also relates to an arrangement of marker sequences containing at least one marker sequence of a cDNA selected from the group SEQ ID No. 92 to 182 and 294 to 313 or in each case a protein coded thereby. The arrangement preferably contains at least 2 to 5 or 10, preferably 30 to 50 marker sequences or 50 to 100 or more marker sequences.

Within the scope of this invention, “arrangement” is synonymous with “array”, and, if this “array” is used to identify substances to be bound on marker sequences, this is to be understood to be an “assay” or a diagnostic device. In a preferred embodiment the arrangement is designed such that the marker sequences represented on the arrangement are present in the form of a grid on a solid support. Furthermore, those arrangements are preferred that permit a high-density arrangement of marker sequences, and the marker sequences are spotted. Such high-density spotted arrangements are disclosed for example in WO 99/57311 and WO 99/57312 and can be used advantageously in a robot-assisted automated high-throughput method.

Within the scope of this invention, however, the term “assay” or diagnostic device likewise comprises those embodiments of a device such as ELISA (for example individual wells of a microtitre plate are coated with the marker sequences or combinations or marker sequences according to the invention, and where appropriate are applied to the individual wells of the microtitre plate in a robot-assisted manner; examples include diagnostic ELISA kits from the company Phadia or “Searchlight” Multiplex ELISA kits from the company Pierce/Thermo Fisher Scientific), bead-based assay (spectrally distinguishable bead populations are coated with marker sequences/combinations of marker sequences. The patient sample is incubated with this bead population and bound (auto) antibodies are detected by means of a further fluorescence-labelled secondary antibody or a detection reagent by measuring the fluorescence; for example Borrelia IgG kit or Athena Multilyte from the company Multimetrix), line assay (marker sequences or combinations of marker sequences according to the invention are immobilised in a robot-assisted manner on membranes, which are examined or incubated with the patient sample; example “Euroline” from the company Euroimmun AG), Western Blot (example “Euroline-WB” from the company Euroimmun AG), and immunochromatographic methods (for example what are known as lateral flow immunoassays; marker sequences or combinations of marker sequences are immobilised on test strips (membranes, U.S. Pat. No. 5,714,389 and many others); example “One Step HBsAg” test device from Acon Laboratories) or similar immunological single or multiplex detection methods.

The marker sequences of the arrangement are fixed on a solid support, but are preferably spotted or immobilised or printed on, that is to say applied in a reproducible manner. One or more marker sequences can be present multiple times in the totality of all marker sequences and may be present in different quantities based on a spot. Furthermore, the marker sequences can be standardised on the solid support (for example by means of serial dilution series of, for example, human globulins as internal calibrators for data normalisation and quantitate evaluation).

The invention therefore concerns an assay or protein biochip or one or more beads (bead-based assay) consisting of an arrangement containing marker sequences according to the invention.

In a further embodiment the marker sequences are present as clones. Such clones can be obtained for example by means of a cDNA expression library according to the invention (Büssow et al. 1998 (above)). In a preferred embodiment such expression libraries containing clones are obtained using expression vectors from a cDNA expression library consisting of the cDNA marker sequences. These expression vectors preferably contain inducible promoters. The induction of the expression can be carried out for example by means of an inducer, such as IPTG. Suitable expression vectors are described in Terpe et al. (Terpe T Appl Microbiol Biotechnol. 2003 January; 60(5):523-33). Expression libraries are known to a person skilled in the art; they can be produced in accordance with standard works, such as Sambrook et al, “Molecular Cloning, A laboratory handbook, 2nd edition (1989), CSH press, Cold Spring Harbor, N.Y. Expression libraries that are tissue-specific (for example human tissue, in particular human organs) are furthermore preferable. Further, expression libraries that can be obtained by means of exon-trapping are also included in accordance with the invention. Instead of the term expression library, reference may also be made synonymously to an expression bank.

Protein biochips or beads or corresponding expression libraries that do not exhibit any redundancy (what is known as a Uniclone® library) and that can be produced in accordance with the teaching of WO 99/57311 and WO 99/57312 are furthermore preferred. These preferred Uniclone® libraries have a high proportion of non-defective fully expressed proteins of a cDNA expression library.

Within the scope of this invention the clones can also be, but are not limited to, transformed bacteria, recombinant phages or transformed cells of mammals, insects, fungi, yeasts or plants.

The clones are fixed, spotted or immobilised on a solid support.

The invention therefore relates to an arrangement, wherein the marker sequences are present as clones.

In addition, the marker sequences can be present in the respective form of a fusion protein, which for example contains at least one affinity epitope or “tag”. The tag may be or may contain one such as c-myc, his tag, arg tag, FLAG, alkaline phosphatase, V5 tag, T7 tag or strep tag, HAT tag, NusA, S tag, SBP tag, thioredoxin, DsbA, a fusion protein, preferably a cellulose-binding domain, green fluorescent protein, maltose-binding protein, calmodulin-binding protein, glutathione S-transferase or lacZ.

A marker sequence can be composed of a number of individual marker sequences. This may include the cloning of individual fragments to form a large common fragment and the expression of this combined fragment.

In all embodiments, the term “solid support” includes embodiments such as a filter, a membrane, a magnetic or fluorophore-labelled pellet, a silicon wafer, glass, metal, plastic, a chip, a mass spectrometry target or a matrix. However, a filter and beads are preferred in accordance with the invention.

Furthermore, PVDF, nitrocellulose or nylon is preferred as a filter (for example Immobilon P Millipore, Protran Whatman, Hybond N+ Amersham).

In a further preferred embodiment of the arrangement according to the invention, this corresponds to a grid with the dimensions of a microtiter plate (8-12 well strips, 96 wells, 384 wells or more), a silicon wafer, a chip, a mass spectrometry target or a matrix.

In a further preferred embodiment pellets or what are known as beads are used as support. Here, bead-based multiplex assays are preferably used. The analysis and evaluation of the bead-based assays can be performed for example with a Luminex analysis system, which is performed on the basis of the method of flow cytometry with use of two different lasers.

Whereas the measurements on planar protein arrays offer merely a dynamic range of 1.5-2 magnitudes (powers of 10), a dynamic range of 3.5-4 magnitudes can be covered by the use of Luminex beads. The measurements in the low response ranges also provide very good coefficients of variation (CVs), i.e. no more than 10%.

Whereas the measurements on planar protein arrays offer merely coefficients of variation (CVs) from 10 to 25% (intra-array comparison) or 10 to 50% (inter-array comparison), the CVs of the Luminex measurements are located between 3 to 10%. An assay quality not generally achieved by commercial ELISAs is thus provided. The known disadvantages (limited plexing rate by interference of different detection antibodies) for Luminex-based analysis and diagnostic methods do not occur with the UNlarray concept, since merely a single fluorescence-labelled anti-human IgG from goat, sheep or mouse is used as detection probe. Due to the transfer of the UNlarray concept to Luminex (i.e. bead-based protein arrays), a number of apparatuses can additionally be saved, i.e. protein printers, hybridisation machines and array readers, and can be replaced by one apparatus. Here, the UNlarray concept is not bound to Luminex, but can also be used on other platforms, such as Randox, VBC Genomics, etc. The high measurement accuracy and the low CVs of the individual measurements allow the use of better and new statistical methods for the identification of potent individual markers and also for rapid sorting of false positives.

In a further embodiment the invention relates to an assay or protein biochip for identifying and characterising a substance for rheumatoid arthritis, characterised in that an arrangement or assay according to the invention is brought into contact with a.) at least one substance to be examined, and b.) binding success is detected. The substance to be examined may be any native or non-native biomolecule, a synthetic chemical molecule, a mixture, or a substance library. Once the substance to be examined contacts a marker sequence, the binding success is evaluated, this being performed for example with use of commercially available image analysing software (GenePix Pro (Axon Laboratories), Aida (Raytest), ScanArray (Packard Bioscience).

Protein-protein interactions (for example protein at the marker sequence, such as antigen/antibody) or corresponding “means for detecting the binding success” can be visualised for example by means of fluorescence labelling, biotinylation, radio-isotope labelling or colloid gold or latex particle labelling in the conventional manner. Bound antibodies are detected with the aid of secondary antibodies, which are labelled using commercially available reporter molecules (for example Cy, Alexa, Dyomics, FITC or similar fluorescent dyes, colloidal gold or latex particles), or with reporter enzymes, such as alkaline phosphatase, horseradish peroxidase, etc., and the corresponding colorimetric, fluorescent or chemoluminescent substrates. A readout is performed for example by means of a microarray laser scanner, a CCD camera or visually.

In a further embodiment the invention relates to a drug/active agent or prodrug for rheumatoid arthritis, developed and obtainable by the use of the assay or protein biochip according to the invention.

The invention therefore also relates to the use of an arrangement or an assay according to the invention for screening active agents for rheumatoid arthritis.

### EXAMPLES AND FIGURES

FIG. 1: Tables 8 and 8a

FIG. 2: Volcano Plot Rheumatoid Arthritis vs. Control

FIG. 3: Volcano Plot CCP negative in the RA Group vs. Control

### EXAMPLE 1

### Selection of the Marker Sequence Candidates and Production of the Luminex Beads

Ten or more patient samples were screened individually against a cDNA expression library. The rheumatoid arthritis-specific expression clones were determined by a comparison with ten or more healthy samples. The identity of the marker sequences was determined by DNA sequencing.

Differential screening was performed between two protein biochips, one from a cDNA expression bank of a patient and one from a healthy test subject, and the differential clones were detected by means of fluorescence labelling and evaluated by means of bioinformatics.

There were about ˜3,500 proteins which were included in examinations with protein biochips and were apparent as antigens for inflammatory diseases.

These 3,500 proteins were then produced in relatively large quantities (several mg), purified, and coupled on Luminex beads. In addition, biomarkers already known (public domain), such as CCP, for rheumatoid arthritis, Aquaporin 4 for Neuromyolitis Optica, various cytokines, typical autoimmune markers, etc., were produced and measured together with the 3,500 proteins. The inclusion of known autoantigens in screening and validation is important insofar as the best new candidates can be selected very quickly as a result.

### EXAMPLE 2

### Selection of the Patients and Test Subjects for the Validation of the Marker Sequence Candidates

Selection of the group of individuals to be tested: Patients with rheumatoid arthritis (RA) and test subjects without rheumatoid arthritis (control).

TABLE 1
Homogeneity analysis of the groups (patients, test subjects)

| | Group | Attribute | p-value |
|---|---|---|---|
| 1 | RA vs. | Age | 0.3996 |
| | Control | | |
| 2 | RA vs. | Gender | <0.0001 |
| | control | | |

TABLE 2
Statistical data regarding the age of the group

| | | | N | | | | | |
|---|---|---|---|---|---|---|---|---|
| | Group | N | miss | Mean | Median | SD | Min. | Max. |
| 1 | Control | 71 | 0 | 54.62 | 56.00 | 11.32 | 26.00 | 69.00 |
| 2 | RA | 75 | 0 | 56.56 | 57.00 | 13.23 | 25.00 | 89.00 |

TABLE 3
Statistical data regarding the sex of the group

| | Group | Sex | Frequency | Proportion |
|---|---|---|---|---|
| 1 | Control | F | 52 | 73.24 |
| 2 | Control | M | 19 | 26.76 |
| 3 | RA | F | 54 | 72.00 |
| 4 | RA | M | 21 | 28.00 |

TABLE 4
Statistical data for DAS28

| | | | N | | | | | |
|---|---|---|---|---|---|---|---|---|
| | Group | N | miss | Mean | Median | SD | Min. | Max. |
| 1 | Control | 0 | 71 | | | | | |
| 2 | RA | 55 | 20 | 3.28 | 3.00 | 1.14 | 1.60 | 5.70 |

TABLE 5
Statistical data for DAS28 in view of category

| | Group | DAS 28 | Range | Frequency | Proportion |
|---|---|---|---|---|---|
| 1 | Control | Remission | <2.6 | 0 | 0.00 |
| 2 | Control | Low | 2.6-3.1 | 0 | 0.00 |
| 3 | Control | Moderate | >3.2-5.1 | 0 | 0.00 |
| 4 | Control | High | >5.1 | 0 | 0.00 |
| 5 | RA | Remission | <2.6 | 17 | 30.91 |
| 6 | RA | Low | 2.6-3.1 | 13 | 23.64 |
| 7 | RA | Moderate | >3.2-5.1 | 19 | 34.55 |
| 8 | RA | High | >5.1 | 6 | 10.91 |

TABLE 6
Statistical data in respect of the duration
of the illness

| | | | N | | | | | |
|---|---|---|---|---|---|---|---|---|
| | Group | N | miss | Mean | Median | SD | Min. | Max. |
| 1 | Control | 0 | 71 | | | | | |
| 2 | RA | 71 | 4 | 132.20 | 79.36 | 132.50 | 1.38 | 543.40 |

### EXAMPLE 3

### Identification of the Marker Sequences According to the Invention

Table 7 summarises the clone sequences SEQ ID No. 1 to 91 and 274 to 293, which are specific for rheumatoid arthritis and were used for identification of the sequences SEQ ID No. 92 to 273 and 294 to 333 specific for rheumatoid arthritis. The details regarding the sequence data will become clear from the accompanying sequence protocol.

TABLE 7

| SEQ | | | | | | |
|---|---|---|---|---|---|---|
| ID | | Gene | | | | |
| No. | Comparison | ID | Gene Name | Gene Symbol | RefSeq Accession | GI Accession |
| 1 | Control | 8655 | dynein, light | DYNLL1 | ref\|NM_003746 | gi\|4505813 |
| | vs RA | | chain, LC8-type 1 | | | |
| 2 | Control | 440 | asparagine | ASNS | ref\|NM_133436 | gi\|168229248 |
| | vs RA | | synthetase | | | |
| | | | (glutamine- | | | |
| | | | hydrolyzing) | | | |
| 3 | Control | 79703 | chromosome 11 | C11orf80 | ref\|NM_024650 | gi\|170932471 |
| | vs RA | | open reading | | | |
| | | | frame 80 | | | |
| 4 | Control | 977 | CD151 molecule | CD151 | ref\|NM_004357 | gi\|21237748 |
| | vs RA | | (Raph blood | | | |
| | | | group) | | | |
| 5 | Control | 896 | cyclin D3 | CCND3 | ref\|NM_001760 | gi\|4502619 |
| | vs RA | | | | | |
| 6 | Control | 309 | annexin A6 | ANXA6 | ref\|NM_004033 | gi\|71773415 |
| | vs RA | | | | | |
| 7 | Control | 440354 | PI-3-kinase- | LOC440354 | | gi\|194385888 |
| | vs RA | | related kinase | | | |
| | | | SMG-1 | | | |
| | | | pseudogene | | | |
| 8 | Control | 5909 | RAP1GAP RAP1 | RAP1GAP | ref\|NM_002885 | gi\|4506415 |
| | vs RA | | GTPase | | | |
| | | | activating | | | |
| | | | protein [Homo | | | |
| | | | sapiens] | | | |
| 9 | Control | 8751 | ADAM | ADAM15 | ref\|NM_207196 | gi\|46909598 |
| | vs RA | | metallopeptidas | | | |
| | | | domain 15 | | | |
| 10 | Control | 1175 | adaptor-related | AP2S1 | ref\|NM_004069 | gi\|70906430 |
| | vs RA | | protein complex | | | |
| | | | 2, sigma 1 | | | |
| | | | subunit | | | |
| 11 | Control | 326625 | methylmalonic | MMAB | ref\|NM_052845 | gi\|16418349 |
| | vs RA | | aciduria | | | |
| | | | (cobalamin | | | |
| | | | deficiency) cbIB | | | |
| | | | type | | | |
| 12 | Control | 9129 | PRP3 pre-mRNA | PRPF3 | ref\|NM_004698 | gi\|4758556 |
| | vs RA | | processing | | | |
| | | | factor 3 | | | |
| | | | homolog (S. | | | |
| | | | cerevisiae) | | | |
| 13 | Control | 23170 | tubulin tyrosine | TTLL12 | ref\|NM_015140 | gi\|11056036 |
| | vs RA | | ligase-like | | | |
| | | | family, member | | | |
| | | | 12 | | | |
| 14 | Control | 3098 | hexokinase 1 | HK1 | ref\|NM_033500 | gi\|188497750 |
| | vs RA | | | | | |
| 15 | Control | 81620 | chromatin | CDT1 | ref\|NM_030928 | gi\|188497689 |
| | vs RA | | licensing and | | | |
| | | | DNA replication | | | |
| | | | factor 1 | | | |
| 16 | Control | 8140 | solute carrier | SLC7A5 | ref\|NM_003486 | gi\|71979932 |
| | vs RA | | family 7 (amino | | | |
| | | | acid transporter | | | |
| | | | light chain, L | | | |
| | | | system), | | | |
| | | | member 5 | | | |
| 17 | Control | 27344 | proprotein | PCSK1N | ref\|NM_013271 | gi\|7019519 |
| | | | convertase | | | |
| | | | subtilisin/kexin | | | |
| | | | type 1 inhibitor | | | |
| 18 | Control | 727910 | TLC domain | TLCD2 | | gi\|205830928 |
| | vs RA | | containing 2 | | | |
| 19 | Control | 5819 | poliovirus | PVRL2 | ref\|NM_001042724 | gi\|112789532 |
| | vs RA | | receptor-related | | | |
| | | | 2 (herpesvirus | | | |
| | | | entry mediator B) | | | |
| 20 | Control | 84196 | ubiquitin | USP48 | ref\|NM_032236 | gi\|52630449 |
| | vs RA | | specific | | | |
| | | | peptidase 48 | | | |
| 21 | Control | 22913 | RNA binding | RALY | ref\|NM_016732 | gi\|8051631 |
| | vs RA | | protein, | | | |
| | | | autoantigenic | | | |
| | | | (hnRNP- | | | |
| | | | associated with | | | |
| | | | lethal yellow | | | |
| | | | homolog | | | |
| | | | (mouse)) | | | |
| 22 | Control | 64221 | roundabout, | ROBO3 | ref\|NM_022370 | gi\|48476182 |
| | vs RA | | axon guidance | | | |
| | | | receptor, | | | |
| | | | homolog 3 | | | |
| | | | (Drosophila) | | | |
| 23 | Control | 728294 | D-2-hydroxyglutarate | D2HGDH | ref\|NM_152783 | gi\|119964728 |
| | vs RA | | dehydrogenase | | | |
| 24 | Control | 79921 | transcription | TCEAL4 | ref\|NM_001006935 | gi\|55749442 |
| | vs RA | | elongation | | | |
| | | | factor A (SII)-like 4 | | | |
| 25 | Control | 147808 | zinc finger | ZNF784 | ref\|NM_203374 | gi\|42794622 |
| | vs RA | | protein 784 | | | |
| 26 | Control | 9040 | UBE2M | UBE2M | ref\|NM_003969 | gi\|150417997 |
| | vs RA | | ubiquitin- | | | |
| | | | conjugating | | | |
| | | | enzyme E2M [Homo | | | |
| | | | sapiens] | | | |
| 27 | Control | 55778 | ZNF839 zinc | ZNF839 | ref\|NM_018335 | gi\|153251839 |
| | vs RA | | finger protein | | | |
| | | | 839 [Homo | | | |
| | | | sapiens] | | | |
| 28 | Control | 27344 | proprotein | PCSK1N | ref\|NM_013271 | gi\|7019519 |
| | vs RA | | convertase | | | |
| | | | subtilisin/kexin | | | |
| | | | type 1 inhibitor | | | |
| 29 | Control | 10726 | nuclear | NUDC | ref\|NM_006600 | gi\|5729953 |
| | vs RA | | distribution | | | |
| | ccp neg | | gene C homolog | | | |
| | | | (A. nidulans) | | | |
| 30 | Control | 9890 | lipid phosphate | LPPR4 | ref\|NM_014839 | gi\|33636722 |
| | vs RA | | phosphatase- | | | |
| | ccp neg | | related protein | | | |
| | | | type 4 | | | |
| 31 | Control | 1794 | dedicator of | DOCK2 | ref\|NM_004946 | gi\|31377468 |
| | vs RA | | cytokinesis 2 | | | |
| | ccp neg | | | | | |
| 32 | Control | 3931 | lecithin- | LCAT | ref\|NM_000229 | gi\|4557892 |
| | vs RA | | cholesterol | | | |
| | ccp neg | | acyltransferase | | | |
| 33 | Control | 58513 | epidermal | EPS15L1 | ref\|NM_021235 | gi\|10864047 |
| | vs RA | | growth factor | | | |
| | ccp neg | | receptor | | | |
| | | | pathway | | | |
| | | | substrate 15- | | | |
| | | | like 1 | | | |
| 34 | Control | 1513 | cathepsin K | CTSK | ref\|NM_000396 | gi\|4503151 |
| | vs RA | | | | | |
| | ccp neg | | | | | |
| 35 | Control | 64434 | nucleolar | NOM1 | ref\|NM_138400 | gi\|61097912 |
| | vs RA | | protein with | | | |
| | ccp neg | | MIF4G domain 1 | | | |
| 36 | Control | 203068 | tubulin, beta | TUBB | ref\|NM_178014 | gi\|29788785 |
| | vs RA | | | | | |
| | ccp neg | | | | | |
| 37 | Control | 119504 | anaphase | ANAPC16 | ref\|NM_173473 | gi\|27735039 |
| | vs RA | | promoting | | | |
| | ccp neg | | complex subunit | | | |
| | | | 16 | | | |
| 38 | Control | 59277 | netrin 4 | NTN4 | ref\|NM_021229 | gi\|93204871 |
| | vs RA | | | | | |
| | ccp neg | | | | | |
| 39 | Control | 6232 | ribosomal | RPS27 | ref\|NM_001030 | gi\|4506711 |
| | vs RA | | protein S27 | | | |
| | ccp neg | | | | | |
| 40 | Control | 23151 | GRAM domain | GRAMD4 | ref\|NM_015124 | gi\|67763814 |
| | vs RA | | containing 4 | | | |
| | ccp neg | | | | | |
| 41 | Control | 6189 | ribosomal | RPS3A | ref\|NM_001006 | gi\|4506723 |
| | vs RA | | protein S3A | | | |
| | ccp neg | | | | | |
| 42 | Control | 6432 | serine/arginine- | SRSF7 | ref\|NM_001031684 | gi\|72534660 |
| | vs RA | | rich splicing | | | |
| | ccp neg | | factor 7 | | | |
| 43 | Control | 7874 | ubiquitin | USP7 | ref\|NM_003470 | gi\|150378533 |
| | vs RA | | specific | | | |
| | ccp neg | | peptidase 7 | | | |
| | | | (herpes virus- | | | |
| | | | associated) | | | |
| 44 | Control | 6418 | SET nuclear | SET | ref\|NM_003011 | gi\|170763498 |
| | vs RA | | oncogene | | | |
| | ccp neg | | | | | |
| 45 | Control | 11258 | dynactin 3 (p22) | DCTN3 | ref\|NM_007234 | gi\|6005745 |
| | vs RA | | | | | |
| | ccp neg | | | | | |
| 46 | Control | 9733 | squamous cell | SART3 | ref\|NM_014706 | gi\|7661952 |
| | vs RA | | carcinoma | | | |
| | ccp neg | | antigen | | | |
| | | | recognized by T | | | |
| | | | cells 3 | | | |
| 47 | Control | 5217 | profilin 2 | PFN2 | ref\|NM_053024 | gi\|16753215 |
| | vs RA | | | | | |
| | ccp neg | | | | | |
| 48 | Control | 302 | annexin A2 | ANXA2 | ref\|NM_004039 | gi\|4757756 |
| | vs RA | | | | | |
| | ccp neg | | | | | |
| 49 | Control | 55830 | glycosyltransfer | GLT8D1 | ref\|NM_018446 | gi\|8923855 |
| | vs RA | | ase 8 domain | | | |
| | ccp neg | | containing 1 | | | |
| 50 | Control | 3557 | interleukin 1 | IL1RN | ref\|NM_000577 | gi\|10835147 |
| | vs RA | | receptor | | | |
| | ccp neg | | antagonist | | | |
| 51 | Control | 90809 | transmembrane | TMEM55B | ref\|NM_144568 | gi\|154816184 |
| | vs RA | | protein 55B | | | |
| | ccp neg | | | | | |
| 52 | Control | 163033 | zinc finger | ZNF579 | ref\|NM_152600 | gi\|110681708 |
| | vs RA | | protein 579 | | | |
| | ccp neg | | | | | |
| 53 | Control | 9605 | chromosome 16 | C16orf7 | ref\|NM_004913 | gi\|108860690 |
| | vs RA | | open reading | | | |
| | ccp neg | | frame 7 | | | |
| 54 | Control | 6710 | spectrin, beta, | SPTB | ref\|NM_001024858 | gi\|67782321 |
| | vs RA | | erythrocytic | | | |
| | ccp neg | | | | | |
| 55 | Control | 2934 | gelsolin | GSN | ref\|NM_198252 | gi\|38044288 |
| | vs RA | | | | | |
| | ccp neg | | | | | |
| 56 | Control | 4591 | tripartite motif | TRIM37 | ref\|NM_015294 | gi\|15147333 |
| | vs RA | | containing 37 | | | |
| | ccp neg | | | | | |
| 57 | Control | 6903 | tubulin folding | TBCC | ref\|NM_003192 | gi\|4507373 |
| | vs RA | | cofactor C | | | |
| | ccp neg | | | | | |
| 58 | Control | 11140 | cell division | CDC37 | ref\|NM_007065 | gi\|5901922 |
| | vs RA | | cycle 37 | | | |
| | ccp neg | | homolog (S. | | | |
| | | | cerevisiae) | | | |
| 59 | Control | 7458 | eukaryotic | EIF4H | ref\|NM_031992 | gi\|14702180 |
| | vs RA | | translation | | | |
| | ccp neg | | initiation factor 4H | | | |
| 60 | Control | 6687 | spastic paraplegia 7 | SPG7 | ref\|NM_003119 | gi\|4507173 |
| | vs RA | | (pure and | | | |
| | ccp neg | | complicated | | | |
| | | | autosomal | | | |
| | | | recessive) | | | |
| 61 | Control | 22902 | RUN and FYVE | RUFY3 | ref\|NM_014961 | gi\|7662352 |
| | vs RA | | domain containing 3 | | | |
| | ccp neg | | | | | |
| 62 | Control | 6144 | ribosomal | RPL21 | ref\|NM_000982 | gi\|18104948 |
| | vs RA | | protein L21 | | | |
| | ccp neg | | | | | |
| 63 | Control | 84893 | F-box protein, | FBXO18 | ref\|NM_178150 | gi\|30795119 |
| | vs RA | | helicase, 18 | | | |
| | ccp neg | | | | | |
| 64 | Control | 10295 | branched chain | BCKDK | ref\|NM_005881 | gi\|171906589 |
| | vs RA | | ketoacid | | | |
| | ccp neg | | dehydrogenase | | | |
| | | | kinase | | | |
| 65 | Control | 3980 | ligase III, DNA, | LIG3 | ref\|NM_002311 | gi\|173747844 |
| | vs RA | | ATP-dependent | | | |
| | ccp neg | | | | | |
| 66 | Control | 3913 | laminin, beta 2 | LAMB2 | ref\|NM_002292 | gi\|1119703755 |
| | vs RA | | (laminin S) | | | |
| | ccp neg | | | | | |
| 67 | Control | 221061 | family with | FAM171A1 | ref\|NM_001010924 | gi\|163025206 |
| | vs RA | | sequence | | | |
| | ccp neg | | similarity 171, | | | |
| | | | member A1 | | | |
| 68 | Control | 790 | carbamoyl- | CAD | ref\|NM_004341 | gi\|18105007 |
| | vs RA | | phosphate | | | |
| | ccp neg | | synthetase 2, | | | |
| | | | aspartate | | | |
| | | | transcarbamylase, | | | |
| | | | and dihydroorotase | | | |
| 69 | Control | 8891 | eukaryotic | EIF2B3 | ref\|NM_020365 | gi\|19966779 |
| | vs RA | | translation | | | |
| | ccp neg | | initiation factor | | | |
| | | | 2B, subunit 3 | | | |
| | | | gamma, 58 kDa | | | |
| 70 | Control | 23367 | La | LARP1 | ref\|NM_015315 | gi\|139725634 |
| | vs RA | | ribonucleoprote | | | |
| | ccp neg | | in domain | | | |
| | | | family, member 1 | | | |
| 71 | Control | 3913 | laminin, beta 2 | LAMB2 | ref\|NM_002292 | gi\|19703755 |
| | vs RA | | (laminin S) | | | |
| | ccp neg | | | | | |
| 72 | Control | 4000 | lamin A/C | LMNA | ref\|NM_170707 | gi\|27436946 |
| | vs RA | | | | | |
| | ccp neg | | | | | |
| 73 | Control | 9026 | huntingtin | HIP1R | ref\|NM_003959 | gi\|48762942 |
| | vs RA | | interacting | | | |
| | ccp neg | | protein 1 | | | |
| | | | related | | | |
| 74 | Control | 23608 | makorin ring | MKRN1 | | gi\|1119604358 |
| | vs RA | | finger protein 1 | | | |
| | ccp neg | | | | | |
| 75 | Control | 51585 | PCF11, cleavage | PCF11 | ref\|NM_015885 | gi\|133620745 |
| | vs RA | | and | | | |
| | ccp neg | | polyadenylation | | | |
| | | | factor subunit, | | | |
| | | | homolog (S. | | | |
| | | | cerevisiae) | | | |
| 76 | Control | 10808 | heat shock | HSPH1 | ref\|NM_006644 | gi\|42544159 |
| | vs RA | | 105 kDa/110 kDa | | | |
| | ccp neg | | protein 1 | | | |
| 77 | Control | 5716 | proteasome | PSMD10 | ref\|NM_002814 | gi\|4506217 |
| | vs RA | | (prosome, | | | |
| | ccp neg | | macropain) 26S | | | |
| | 9026 | | subunit, non- | | | |
| | | | ATPase, 10 | | | |
| 78 | Control | 57662 | calmodulin | CAMSAP3 | ref\|NM_020902 | gi\|130502140 |
| | vs RA | | regulated | | | |
| | ccp neg | | spectrin- | | | |
| | | | associated | | | |
| | | | protein family, | | | |
| | | | member 3 | | | |
| 79 | Control | 23608 | makorin ring | MKRN1 | ref\|NM_013446 | gi\|223468619 |
| | vs RA | | finger protein 1 | | | |
| | ccp neg | | | | | |
| 80 | Control | 440354 | PI-3-kinase- | LOC440354 | AK304513 | gi\|194385888 |
| | vs RA | | related kinase | | | |
| | | | SMG-1 | | | |
| | | | pseudogene | | | |
| 81 | Control | 727910 | TLC domain | TLCD2 | ref\|NM_001164407 | gi\|256542293 |
| | vs RA | | containing 2 | | | |
| 82 | Control | 9322 | thyroid | TRIP10 | ref\|NM_004240 | gi\|11342676 |
| | vs RA | | hormone | | | |
| | | | receptor | | | |
| | | | interactor 10 | | | |
| 83 | Control | 9516 | lipopolysacchari | LITAF | ref\|NM_004862 | gi\|165787265 |
| | vs RA | | de-induced TNF | | | |
| | | | factor | | | |
| 84 | Control | 9815 | G protein-coupled | GIT2 | ref\|NM_057169 | gi\|17149830 |
| | vs RA | | receptor kinase | | | |
| | | | interacting | | | |
| | | | ArfGAP 2 | | | |
| 85 | Control | 79643 | chromatin | CHMP6 | ref\|NM_024591 | gi\|31542673 |
| | vs RA | | modifying | | | |
| | | | protein 6 | | | |
| 86 | Control | 23635 | single-stranded | SSBP2 | ref\|NM_012446 | gi\|40787999 |
| | vs RA | | DNA binding | | | |
| | | | protein 2 | | | |
| 87 | Control | 57176 | valyl-tRNA | VARS2 | ref\|NM_020442 | gi\|155741845 |
| | vs RA | | synthetase 2, | | | |
| | | | mitochondrial | | | |
| | | | (putative) | | | |
| 88 | Control | 3190 | heterogeneous | HNRNPK | ref\|NM_031263 | gi\|14165437 |
| | vs RA | | nuclear | | | |
| | | | ribonucleoprote | | | |
| | | | in K | | | |
| 89 | Control | 375 | ADP- | ARF1 | ref\|NM_001658 | gi\|4502201 |
| | vs RA | | fibosylation | | | |
| | | | factor 1 | | | |
| 90 | Control | 4122 | mannosidase, | MAN242 | ref\|NM_006122 | gi\|51477716 |
| | vs RA | | alpha, class 2A, | | | |
| | | | member 2 | | | |
| 91 | Control | 54522 | ankyrin repeat | ANKRD16 | ref\|NM_019046 | gi\|58331111 |
| | vs RA | | domain 16 | | | |
| 274 | Control | 1211 | clathrin, light | CLTA | ref\|NM_007096 | gi\|6005993 |
| | ccp | | chain A | | | |
| | neg/Control | | | | | |
| | vs RA | | | | | |
| 275 | Control | 4898 | nardilysin | NRD1 | ref\|NM_001101662 | gi\|156071452 |
| | vs RA | | (Narginine dibasic | | | |
| | | | covertase) | | | |
| 276 | Control | 5425 | polymerase | POLD2 | ref\|NM_006230 | gi\|379056381 |
| | vs RA | | (DNA directed), | | | |
| | ccp neg | | delta 2, | | | |
| | | | accessory | | | |
| | | | subunit | | | |
| 277 | Control | 27289 | Rho family | RND1 | ref\|NM_014470 | gi\|7657514 |
| | vs RA | | GTPase 1 | | | |
| 278 | Control | 51343 | fizzy/cell | FZR1 | ref\|NM_01136197 | gi\|209969678 |
| | vs RA | | divison cycle 20 | | | |
| | ccp | | related 1 | | | |
| | neg/Control | | (Drosophila) | | | |
| | vs RA | | | | | |
| 279 | Control | 10381 | tubulin, beta 3 | TUBB3 | ref\|NM_006086 | gi\|50592996 |
| | vs RA | | class III | | | |
| 280 | Control | 3799 | kinesin family | KIF5B | ref\|NM_004521 | gi\|4758648 |
| | vs RA | | member 5B | | | |
| 281 | Control | 1152 | creatine kinase, | CKB | ref\|NM_001823 | gi\|21536286 |
| | vs RA | | brain | | | |
| 282 | Control | 7552 | zinc finger | ZNF711 | ref\|NM_021998 | gi\|68348723 |
| | vs RA | | protein 711 | | | |
| 283 | Control | 8682 | phosphoprotein | PEA15 | ref\|NM_003768 | gi\|4505705 |
| | vs RA | | enriched in | | | |
| | | | astrocytes 15 | | | |
| 284 | Control | 57498 | kinase D- | KIDINS220 | ref\|NM_020738 | gi\|55741641 |
| | vs RA | | interacting | | | |
| | | | substrate, | | | |
| | | | 220 kDa | | | |
| 285 | Control | 10038 | poly (ADP-ribose) | PARP2 | ref\|NM_001042618 | gi\|110825963 |
| | vs RA | | polymerase 2 | | | |
| | ccp neg | | | | | |
| 286 | Control | 23474 | ethylmalonic | ETHE1 | ref\|NM_014297 | gi\|41327741 |
| | vs RA | | encephalopathy 1 | | | |
| | ccp neg | | | | | |
| 287 | Control | 1509 | cathepsin D | CTSD | ref\|NM_001909 | gi\|4503143 |
| | vs RA | | | | | |
| | ccp neg | | | | | |
| 288 | Control | 3303 | heat shock | HSPA1A | ref\|NM_005345 | gi\|194248072 |
| | vs RA | | 70 kDa protein 1A | | | |
| | ccp neg | | | | | |
| 289 | Control | 79140 | coiled-coil | CDDC28B | ref\|NM_024296 | gi\|110349769 |
| | vs RA | | domain | | | |
| | | | containing 28B | | | |
| 290 | Control | 6196 | ribsosomal | RPS6KA2 | ref\|NM_021135 | gi\|19923570 |
| | vs RA | | protein S6 | | | |
| | | | kinase, 90 kDa, | | | |
| | | | polypeptide 2 | | | |
| 291 | Control | 64943 | 5′-nucleotidase | NT5DC2 | ref\|NM_022908 | gi\|12597653 |
| | vs RA | | domain | | | |
| | ccp neg | | containing 2 | | | |
| 292 | Control | 25796 | 6-phosphoglucon | PGLS | ref\|NM_012088 | gi\|6912586 |
| | vs RA | | olactonase | | | |
| 293 | Control | 55684 | RAB, member | RABL6 | ref\|NM_024718 | gi\|186700623 |
| | vs RA | | RAS oncogene | | | |
| | | | family-like 6 | | | |

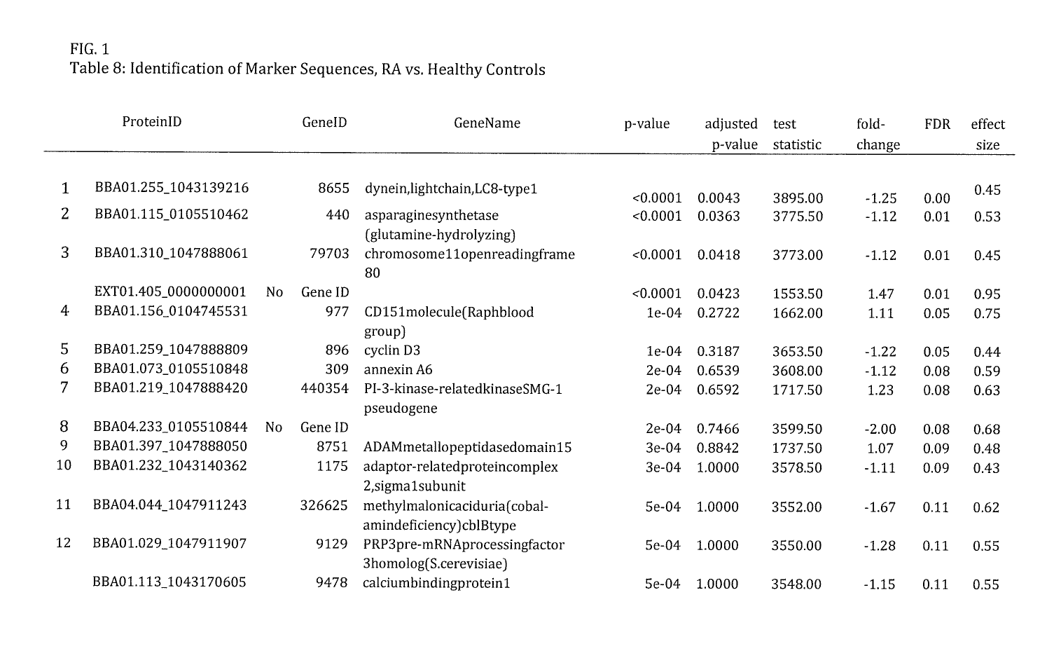

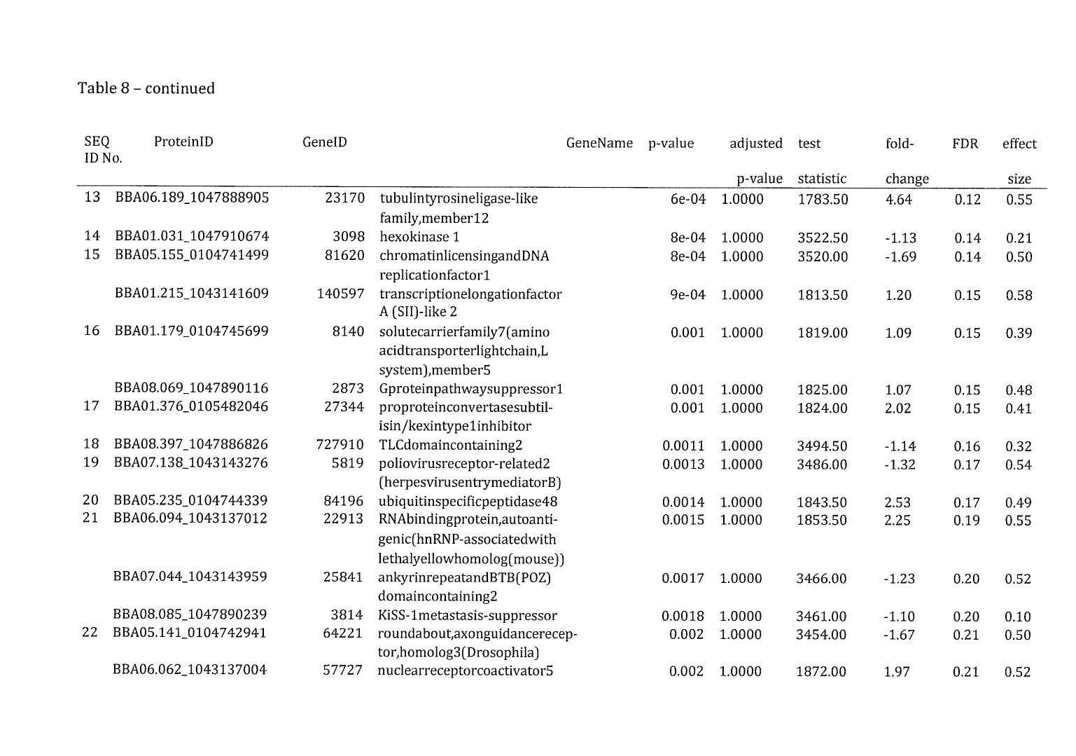

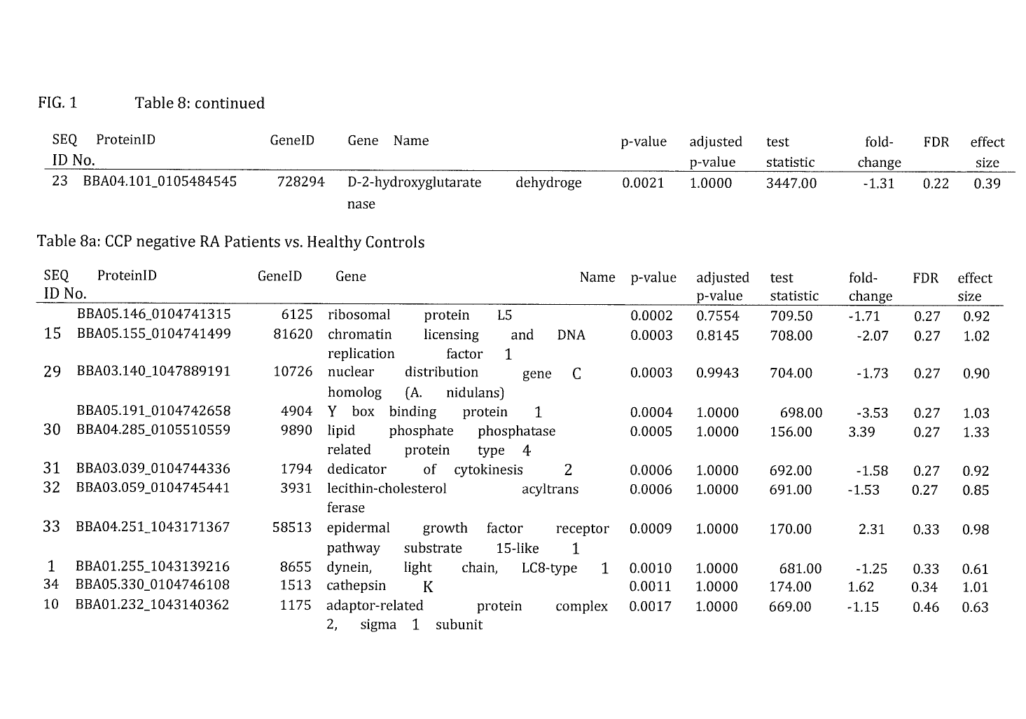

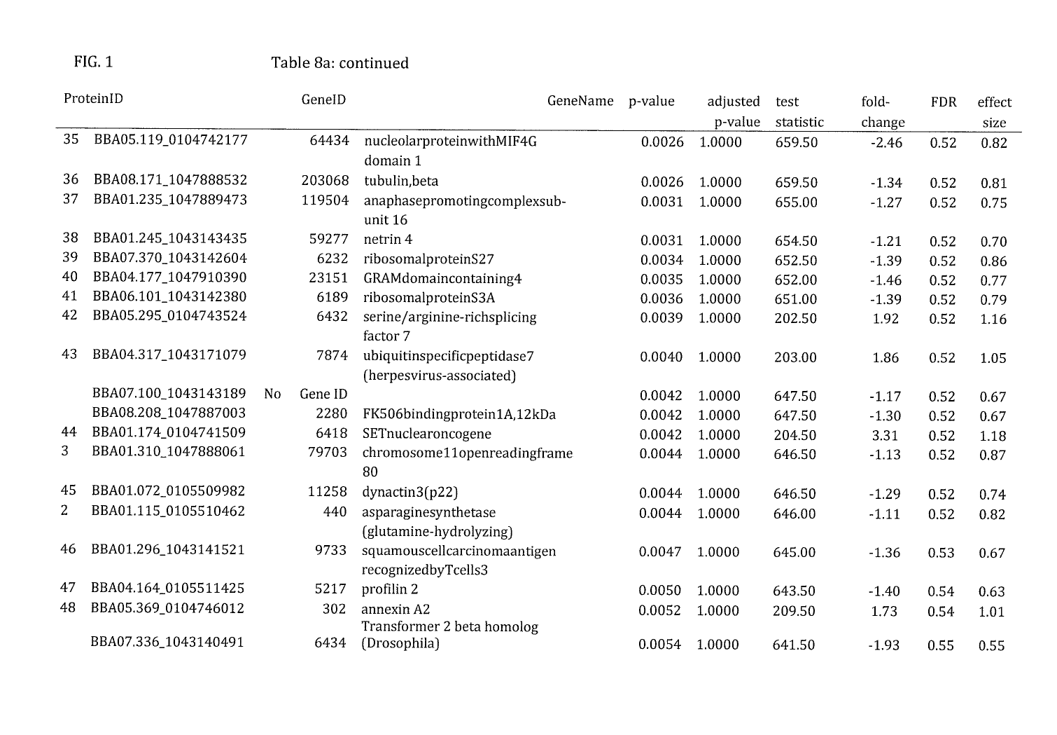

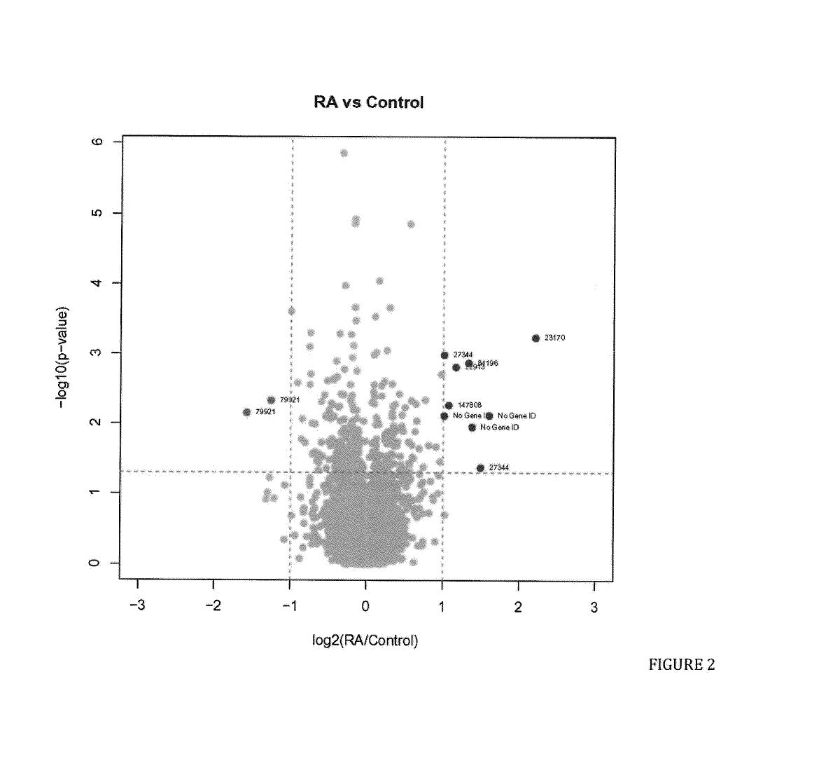

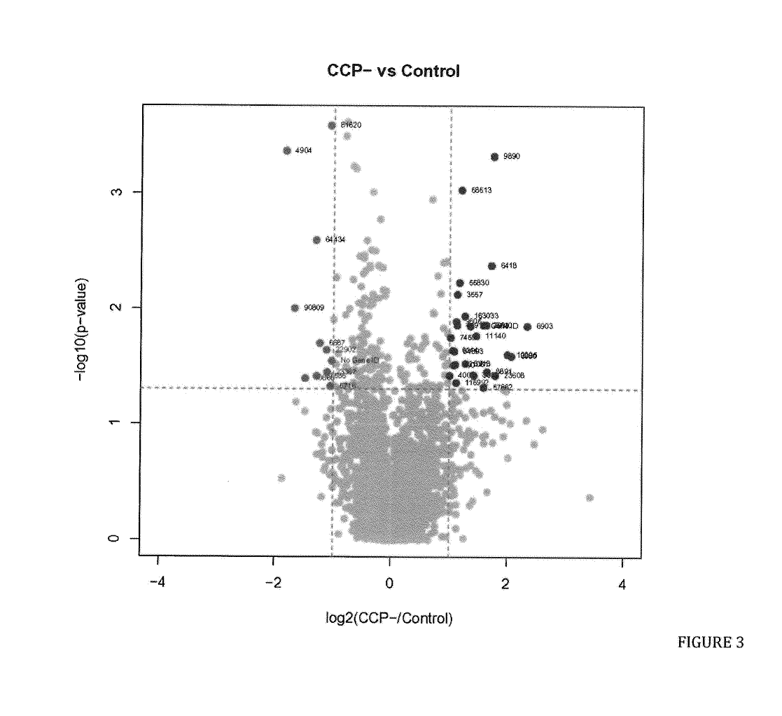

The statistical results regarding the validated marker sequence candidates (marker sequences according to the invention for rheumatoid arthritis) are specified in Tables 8 and 8a (FIG. 1).

1.-15. (canceled)

16. A method for identifying marker sequences for rheumatoid arthritis (RA) comprising the steps of:
a) selecting marker sequence candidates by screening protein biochips representing a cDNA expression library with at least 10 patient samples and at least 10 samples of healthy individuals, wherein each sample is measured individually and marker sequence candidates for RA are selected by a comparison of the results of the screens obtained with the RA patient samples and the results of the screens obtained with the samples from healthy individuals,
b) producing the proteins and/or partial proteins (peptides) coded by the marker sequence candidates by expression of the cDNA of the marker sequence candidates,
c) producing beads to which one or more of the proteins and/or partial proteins (peptides) produced in step b) are coupled,
d) validating the marker sequence candidates coupled to the beads by means of samples from patients with RA and samples from healthy individuals in that marker sequences for RA demonstrate an interaction with the samples from patients with RA and demonstrate a comparatively lower or no interaction with the samples from healthy individuals, wherein the marker sequences SEQ ID No. 1 to 182, SEQ ID. No. 183 to 273, SEQ ID No. 274 to 313 and SEQ ID No. 314 to SEQ ID No. 333 are obtained.

17. The method according to claim 16, wherein an interaction between marker sequence candidate and the samples in method step d) is detected by means of a fluorescence signal, wherein the intensity of the fluorescence signal correlates with the intensity of the interaction.

18. The method according to claim 16, characterised in that steps c) and d) are performed in the presence of CCP (cytochrome c peroxidase), wherein the marker sequences SEQ ID No. 29 to 79, SEQ ID No. 120 to 170, SEQ ID No. 211 to 261, SEQ ID No. 274, SEQ ID No. 276, SEQ ID No. 278, SEQ ID No. 285 to 288, SEQ ID No. 291, SEQ ID No. 294, SEQ ID No. 296, SEQ ID No. 298, SEQ ID No. 305 to 308, SEQ ID No. 311, SEQ ID No. 314, SEQ ID No. 316, SEQ ID No. 318, SEQ ID No. 325 to 328, and SEQ ID No. 331 are obtained.

19. A marker sequence for rheumatoid arthritis obtainable by a method according to one of claim 16, wherein the marker sequence is selected from the group of sequences SEQ ID No. 1 to 182 and SEQ ID. No. 274 to 313, a sequence homologous to the sequences SEQ ID No. 1 to 182 or SEQ ID No. 274 to 313 or a partial sequence of SEQ ID No. 1 to 182 or SEQ ID. No. 274 to 313 or a protein coded by SEQ ID No. 1 to 182 or SEQ ID. No. 274 to 313 or a protein coded by a partial sequence of SEQ ID No. 1 to 182 or SEQ ID. No. 274 to 313 or a protein coded by a homologous sequence of SEQ ID No. 1 to 182 or SEQ ID. No. 274 to 313 or a genomic sequence comprising one of the sequences SEQ ID No. 1 to 182 or SEQ ID. No. 274 to 313.

20. A marker sequence for rheumatoid arthritis obtainable by a method according to claim 18, wherein the marker sequence is selected from the group of sequences SEQ ID No. 29 to 79, SEQ ID No. 274, SEQ ID No. 276, SEQ ID No. 278, SEQ ID No. 285 to 288, SEQ ID No. 291 or SEQ ID. No. 120 to 170, SEQ ID No. 294, SEQ ID No. 296, SEQ ID No. 298, SEQ ID No. 305 to 308, SEQ ID No. 311, a sequence homologous to the sequences SEQ ID No. 29 to 79, SEQ ID No. 274, SEQ ID No. 276, SEQ ID No. 278, SEQ ID No. 285 to 288, SEQ ID No. 291 or SEQ ID No. 120 to 170, SEQ ID No. 294, SEQ ID No. 296, SEQ ID No. 298, SEQ ID No. 305 to 308, SEQ ID No. 311 or a partial sequence of SEQ ID No. 29 to 79, SEQ ID No. 274, SEQ ID No. 276, SEQ ID No. 278, SEQ ID No. 285 to 288, SEQ ID No. 291 or SEQ ID. No. 120 to 170, SEQ ID No. 294, SEQ ID No. 296, SEQ ID No. 298, SEQ ID No. 305 to 308, SEQ ID No. 311 or a protein coded by SEQ ID No. 29 to 79, SEQ ID No. 274, SEQ ID No. 276, SEQ ID No. 278, SEQ ID No. 285 to 288, SEQ ID No. 291 or SEQ ID. No. 120 to 170, SEQ ID No. 294, SEQ ID No. 296, SEQ ID No. 298, SEQ ID No. 305 to 308, SEQ ID No. 311 or a protein coded by a partial sequence of SEQ ID No. 29 to 79, SEQ ID No. 274, SEQ ID No. 276, SEQ ID No. 278, SEQ ID No. 285 to 288, SEQ ID No. 291 or SEQ ID. No. 120 to 170, SEQ ID No. 294, SEQ ID No. 296, SEQ ID No. 298, SEQ ID No. 305 to 308, SEQ ID No. 311 or a protein coded by a homologous sequence of SEQ ID No. 29 to 79, SEQ ID No. 274, SEQ ID No. 276, SEQ ID No. 278, SEQ ID No. 285 to 288, SEQ ID No. 291 or SEQ ID. No. 120 to 170, SEQ ID No. 294, SEQ ID No. 296, SEQ ID No. 298, SEQ ID No. 305 to 308, SEQ ID No. 311 or a genomic sequence comprising one of the sequences SEQ ID No. 29 to 79, SEQ ID No. 274, SEQ ID No. 276, SEQ ID No. 278, SEQ ID No. 285 to 288, SEQ ID No. 291 or SEQ ID. No. 120 to 170, SEQ ID No. 294, SEQ ID No. 296, SEQ ID No. 298, SEQ ID No. 305 to 308, or SEQ ID No. 311.

21. Use of one or more marker sequence(s) according to claim 19 for the diagnosis of rheumatoid arthritis, wherein the marker sequence(s) is/are determined on or from a patient to be examined.

22. The use according to claim 21, characterised in that 2 or 3, preferably 4 or 5, particularly preferably 6, 7 or 8 or more different marker sequences, for example 10 to 20 or 30 or more different marker sequences are determined on or from a patient to be examined.

23. The use according to one of claim 21, characterised in that the marker sequence(s) is/are applied to a solid support, wherein the solid support is selected from filters, membranes, wafers, for example silicon wafers, glass, metal, plastic, chips, mass spectrometry targets, matrices, and beads, for example magnetic, coated or labelled beads, such as fluorophore-labelled beads or Luminex beads.

24. A method for diagnosing rheumatoid arthritis, wherein
a.) at least one marker sequence according to claim 19 is applied to a solid support, preferably to a bead, and
b.) is brought into contact with bodily fluid or tissue sample of a patient and
c.) an interaction of the bodily fluid or of the tissue sample with the marker sequence from a.) is detected.

25. A method for stratification, in particular for risk stratification, or for therapy management of a patient with rheumatoid arthritis, wherein at least one marker sequence according to claim 19 is used in order to examine a sample from the patient.

26. An arrangement comprising one or more marker sequence(s) according to claim 19.

27. An assay or protein array comprising an arrangement according to claim 26.

28. Use of an arrangement according to claim 26 for identifying and/or characterising a substance for rheumatoid arthritis containing means for detecting binding success, characterised in that an arrangement or an assay or protein array is brought into contact with a.) at least one substance to be examined and b.) binding success is detected.

29. Use of a protein array according to claim 27 for identifying and/or characterising a substance for rheumatoid arthritis containing means for detecting binding success, characterised in that an arrangement or an assay or protein array is brought into contact with a.) at least one substance to be examined and b.) binding success is detected.

30. A diagnostic agent for the diagnosis of rheumatoid arthritis containing at least one marker sequence according to claim 19 and where appropriate further auxiliaries and additives.

31. Use of at least one marker sequence selected from the marker sequences according to claim 20 for identifying a sub-group of patients within the group of patients with rheumatoid arthritis, wherein the patients in the sub-group cannot be identified by means of the marker CCP.
